(19) United States
(12) Patent Application Publication
(10) Pub. No.: US 2014/0200465 A1
(43) Pub. Date: Jul. 17, 2014

(54) Monitoring Left Ventricle End Diastolic Pressure

(71) Applicant: Kevin M. McIntyre, Boston, MA (US)

(72) Inventor: Kevin M. McIntyre, Boston, MA (US)

(21) Appl. No.: 13/743,985

(22) Filed: Jan. 17, 2013

(51) Int. Cl. A61B 5/022; A61B 5/00

(52) U.S. Cl. CPC A61B 5/02233; A61B 5/0004; A61B 5/7278
USPC 600/499

(57) ABSTRACT

Apparatus for remotely monitoring the left ventricle end diastolic pressure of a person includes an inflatable band for covering the artery of the person and applying a pre-determined pressure to the artery through the band. A pressure transducer in the inflatable band provides a pressure waveform signal representative of the pressure in the artery covered by the inflatable band. A waveform transmitter transmits the pressure waveform signal to a central location for processing the transmitted signal to provide LVEDP signal representative of the left ventricle end diastolic pressure of the person.

This invention relates in general to monitoring left ventricle end diastolic pressure (LVEDP) noninvasively, and more particularly concerns novel apparatus and techniques for performing the monitoring at a central location through wireless communication with apparatus attached to a person at a remote location.

### BACKGROUND OF THE INVENTION

For background reference is made to U.S. Pat. Nos. 3,776,221, 5,291,895, 6,610,018 and 7,404,800, all of which are incorporated herein by reference, including their respective file histories.

### SUMMARY OF THE INVENTION

According to the invention, there is an inflatable band constructed and arranged to cover the artery of a person being monitored having a transducer responsive to the pressure in the artery to provide a pressure waveform signal representative of the pressure in the artery. The band also includes a pressurization access opening for connection to an external pressure source for pressurizing the band to a predetermined level sufficiently high to allow transduction of the pressure in the artery to provide a pressure waveform signal representative of the pressure in the artery, typically in the range between diastolic and systolic pressure of the patient being monitored. There is also a waveform transmitter for transmitting the pressure waveform signal to a central location, which may be in the band. There may also be a waveform acquirer for receiving and storing the pressure waveform signal, that may be in the band. The band may also include a battery for providing electrical energy to electrical apparatus in the band.

The central location includes data processing apparatus such as disclosed in one or more of the U.S. Patents identified above to provide a signal representative of the LVEDP of the patient being monitored from processing the pressure waveform signal received at the central locations.

Other features, objects and advantages of the invention will become apparent from the following detailed description when read in connection with the accompanying drawing in which:

### BRIEF DESCRIPTION OF DRAWING

FIG. 1 is a pictorial representation of a wrist band around the wrist of a person being monitored; and

FIG. 2 is a block diagram of a system according to the invention.

### DETAILED DESCRIPTION

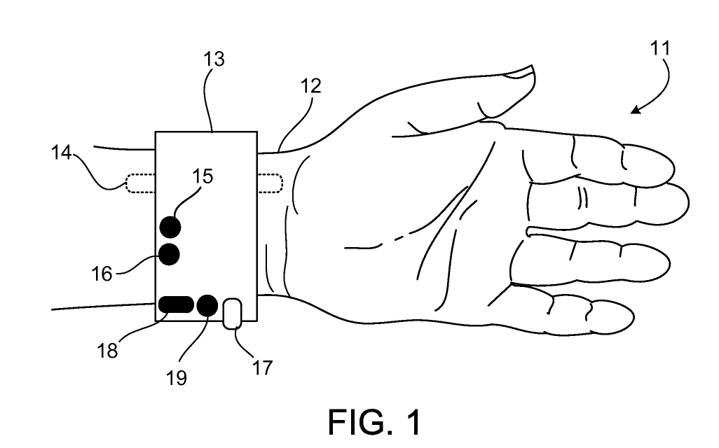

With reference to the drawing and more particularly FIG. 1 thereof, there is shown a pictorial representation of a wrist band on the wrist of a person being monitored. Left hand 11 is shown with its palm forward extending from left wrist 12 surrounded by wrist band 13 covering radial artery 14. Wrist band 13 includes an on-off button 15 for selectively controlling the internal electrical components, such as waveform acquisition unit 16 and waveform transmitter 17 that receive electrical energy from battery 18.

Recorded arterial pressure waveforms are transmitted by waveform transmitter 17 to the central control center where the signal is processed substantially in accordance with the techniques described in U.S. Pat. No. 5,291,895 to provide the LVEDP of the monitored person at the time it was recorded. If the LVEDP is high, the person's caregiver is notified and can prescribe medications, such as Lisinopril, or other procedures to lower the LVEDP of the person to the normal range.

A cuff transducer 13A is positioned over the radial artery to acquire a continuous recording of the pressure waveform signal before during and after a Valsalva maneuver for processing in accordance with the algorithms such as those in the microfiche appendix in the file history of U.S. Pat. No. 5,291,895. The wristband is pressurized to about equal to the monitored persons' diastolic blood pressure established when the person blows into mouth piece 23 to apply a pressure that is nearly equal to the person's diastolic blood pressure.

The monitored person may have an internet connection and a telephone connection to the central monitoring center that is continuously personed. The monitored person may alert the center that an LVEDP measurement has been done and will be forwarded to the center.

The monitored person may have a computer monitor that displays to the person all the steps for completing the test and the e-mail address and telephone number of the center and the person's care provider.

If the person's LVEDP is above normal, it may be transmitted to the person's caregiver for determining whether modifications and treatment or other recommendations should be delivered to the person.

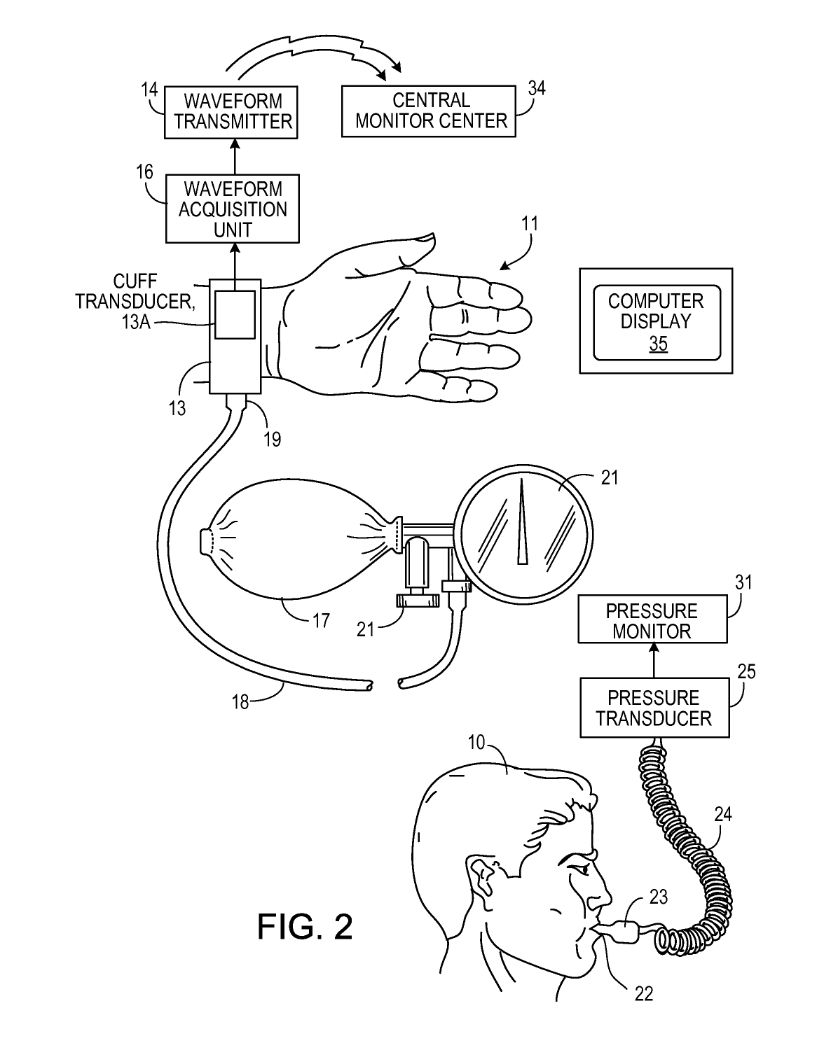

Referring to FIG. 2, there is shown a block-pictorial diagram illustrating the logical arrangement of a system according to the invention. Person 10 blows through mouthpiece 23 and tube 24 to pressure transducer 25 whose pressure is monitored by pressure monitor 31. The monitored person blows into mouthpiece 23 to a pressure of 14-20 mmHG for 8-12 seconds, then remains quiet for 2 minutes until the testing period is over. Wristband 13 is a pressurized cuff that is pressurized through pressure access 19 that may be furnished from a conventional pressurizing source that may be a sphygmomanometer connected to wristband 13 through hose 18 having a valve 21 for closing hose 18 when inflatable wristband 13 is inflated, to apply pressure through pressure transducer 12. Pressure indicator 20 indicates the pressure, preferably substantially the diastolic pressure or substantially above the diastolic pressure (e.g., up to four times diastolic) of person 10 and less than the systolic pressure. The diastolic pressure and systolic pressure for the person may be determined by taking the person's blood pressure in a conventional manner. Cuff transducer 13A in wristband 13 is located over the radial artery 14 to provide a waveform signal that is representative of the pressure waveform produced by blood flowing through radial artery 14. The waveform of the arterial pressure signal sensed by cuff transducer 13A is recorded by waveform acquisition unit 16 for delivery to waveform transmitter 17 for transmission to monitor center 34. Display 35 may display to person 10 steps for person 10 to follow in completing the test, and may also include an email address or telephone number of the central monitor and the person's care provider. The central monitor center 34 receives the waveform signal and can process it in accordance with the apparatus and techniques disclosed in the aforesaid '895 Patent to provide an LVEDP of the monitored person. The LVEDP measurement can then be transmitted back to the person and the person's caregiver.

The invention has a number of advantages. It makes it possible to provide intensive care unit (ICU)-caliber monitoring of LVEDP at any time and at any place where the person is. This advantage can be critically important in that life threatening increases in LVEDP can go unnoticed and lead to death in some person before these life-threatening increases in LVEDP are recognized and treated appropriately.

Conditions in which LVEDP elevations could go unnoticed include ischemic heart disease caused by atherosclerotic obstructions in the coronary arteries. This condition affects many millions of individuals in the US and increases with age. In this condition, ischemia (inadequate blood supply) causes the heart muscle to weaken because of lack of oxygen and the weakness of the heart muscle decreases the amount of blood ejected from the left ventricle with each heartbeat. As a result, LVEDP levels increase abruptly and could be fatal unless they are recognized and treated without delay.

Another condition is unstable hypertension (high blood pressure). In this condition, the pressure of the blood inside the arterial system is higher than normal. Because the heart has to eject blood against a higher pressure with each beat, the amount of blood ejected with each beat may decrease and as a result, the volume of blood in the heart increases, and the LVEDP increases with it. Again, in this condition, the invention can provide timely recognition which can lead to life-saving treatment.

There are other conditions that predispose to left heart failure and abnormal elevations in LVEDP including such serious and common problems as renal failure.

The caregiver may request daily readings or more frequent if the LVEDP is high, such as above 25 mmHG's. If the LVEDP goes above 30, the caregiver may suggest hospitalization.

There has been described novel apparatus and techniques for monitoring the LVEDP of a person remotely from a central location. It is evident that those skilled in the art may now make numerous uses and modifications of and departures from the specific apparatus and techniques herein disclosed without departing from the inventive concepts. Consequently, the invention is to be construed as embracing each and every novel feature and novel combination of features present in or possessed by the apparatus and techniques disclosed herein and limited only by the spirit and scope of the appended claims.

1. Apparatus for monitoring the left ventricle end diastolic pressure of a person comprising,
an inflatable band for covering the artery of a person and applying a predetermined pressure to the artery of the person through the inflatable band,
a pressure transducer in the inflatable band for providing a waveform signal representative of the pressure in the artery covered by the inflatable band when the inflatable band is pressurized at a pressure level within a range for providing a pressure waveform signal representative of the pressure in the artery,
and a waveform transmitter for transmitting the pressure waveform signal to a central location for processing the transmitted signal to provide an LVEDP signal representative of LVEDP of the person being monitored.

2. Apparatus in accordance with claim 1 and further comprising, a waveform acquisition unit for storing the pressure waveform signal coupled to the waveform transmitter to allow selective transmission of the recorded pressure waveform signal to the central location.

3. Apparatus in accordance with claim 1 wherein the waveform transmitter is in the inflatable band.

4. Apparatus in accordance with claim 2 wherein the waveform acquisition unit is in the inflatable band.

5. Apparatus in accordance with claim 1 wherein the inflatable band is formed with an opening for receiving pressurization from a pressure source to maintain the band pressurized at the selected pressure level.

6. A method of monitoring the left ventricle end diastolic pressure of a person remote from a central station comprising,
applying an inflatable band over the artery of a person at the remote location and applying a predetermined pressure to the artery with the inflatable band,
performing a Valsalva maneuver for a predetermined time interval and then resting to establish a pressure waveform representative of the LVEDP of the person,
transmitting the pressure signal waveform to the central location,
receiving the transmitted pressure waveform signal at the central location,
and processing the pressure signal waveform received at the central location to provide a signal representative of the LVEDP of the person.
